(19) United States
(12) Patent Application Publication
(10) Pub. No.: US 2022/0390379 A1
(43) Pub. Date: Dec. 8, 2022

(54) CARTRIDGE-BASED BIOFLUID MONITORING AND ANALYSIS

(71) Applicant: Rajant Health Inc., Malvern, PA (US)

(72) Inventors: Emma K. Murray, Malvern, PA (US); Donald Fiore Schena, Malvern, PA (US); Samuel Elder, Malvern, PA (US); Giana J. Schena, Malvern, PA (US)

(21) Appl. No.: 17/833,842

(22) Filed: Jun. 6, 2022

Related U.S. Application Data
(60) Provisional application No. 63/196,851, filed on Jun. 4, 2021.

(51) Int. Cl. G01N 21/78; B08B 9/08; B08B 9/032

(52) U.S. Cl. CPC G01N 21/78; B08B 9/08; B08B 9/032; B08B 2209/08; B08B 2209/032; G01N 2021/7759

(57) ABSTRACT

A cartridge-based biofluid monitoring system and method of using same are disclosed. The biofluid monitoring system uses a biofluid cartridge configured to be removably inserted into a biofluid cartridge housing. The biofluid cartridge housing can be rotated so that multiple types of analysis can be conducted on one biofluid collection. The biofluid monitoring system may be used at a user's home or at other locations that allow for real-time biofluid monitoring to occur. The biofluid monitoring system allows for point-of-care detection and monitoring of a multitude of clinically relevant biological markers of current or changing health and disease states, by analyzing the user's biofluid.

### CROSS REFERENCE TO RELATED APPLICATION(S)

This application claims priority to U.S. Provisional Patent Application Ser. No. 63/196,851, filed on Jun. 4, 2021, which is hereby incorporated herein by reference in its entirety.

### GOVERNMENT SPONSORSHIP

None

### FIELD OF THE INVENTION

Embodiments are in the field of biofluid monitoring systems. More particularly, embodiments disclosed herein relate to cartridge-based biofluid monitoring systems.

### BACKGROUND OF THE INVENTION

At-home monitoring of urinary output is essential for diagnosis, monitoring, and prevention of disease. Currently, urinary monitoring is only done in clinical settings requiring a prescription. That results in long wait times, the hassle of getting to the appointment, and a long waiting period for test results. Importantly, by the time the information is analyzed, the likelihood that the analysis no longer corresponds to an individual's current health state is very high.

Thus, it is desirable to provide a cartridge-based biofluid monitoring system that is able to overcome the above disadvantages.

Advantages of the present invention will become more fully apparent from the detailed description of the invention hereinbelow.

### SUMMARY OF THE INVENTION

Embodiments are directed to a cartridge-based biofluid monitoring system including: a biofluid cartridge housing; and a biofluid cartridge configured to be removably inserted into the biofluid cartridge housing. The biofluid cartridge includes a storage chamber. The storage chamber is configured to store biofluid.

Embodiments are also directed to a method for using a cartridge-based biofluid monitoring system. The method includes providing a cartridge-based biofluid monitoring system including: a biofluid cartridge housing; and a biofluid cartridge comprising a storage chamber. The method also includes: removably inserting the biofluid cartridge into the biofluid cartridge housing; and storing biofluid in the storage chamber.

Additional embodiments and additional features of embodiments for the cartridge-based biofluid monitoring system are described below.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It is understood, however, that the inventive concepts disclosed herein are not limited to the precise arrangements and instrumentalities shown in the figures. The detailed description will refer to the following drawings in which like numerals, where present, refer to like items.

FIG. 1 is a diagram of an architecture of a digital health ecosystem;

FIG. 2 is a drawing illustrating a schematic view of an exemplary overall workflow design of a biofluid monitoring system from a spectrometer light source, through a sample, to a detector;

FIG. 3A is a drawing illustrating an exemplary partially transparent biofluid monitoring system for sterile collection and analysis of urine;

FIG. 3B is a drawing illustrating a plan view of the biofluid monitoring system shown in FIG. 3A;

FIG. 3C is a drawing illustrating an enlarged perspective view of a portion of the biofluid monitoring system shown in FIG. 3B. The figure shows, inter alia, a tube for transport of biofluid into cartridges, as well as a conduit for the cleaning fluid for post-analysis cleansing;

FIG. 3D is a drawing illustrating a further enlarged view of a portion of the biofluid monitoring system shown in FIG. 3C. The removable cartridges can be installed from above into the rotatable biofluid cartridge housing;

FIG. 3E is a drawing illustrating a plan view of the portion of the biofluid monitoring system shown in FIG. 3D. The spectrometer is positioned in the center of the rotatable biofluid cartridge housing. The spectrometer is in a fixed physical position, and the cartridges are filled with the tube and then rotated and each read by the spectrometer/detector. Following this, the cartridge can be removed and then the system flushed with cleaning fluid from the cleaning fluid container; and

FIG. 3F is a drawing illustrating a further enlarged view of the rotatable biofluid cartridge housing with the pipette to fill the cartridges.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical urinalysis monitoring system or typical method of using a urinalysis monitoring system. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

For purposes of this disclosure, the term “monitoring” is interchangeable with the term “analyzing”. Likewise, the phrase “monitoring system” is interchangeable with the phrase “analysis system”.

For purposes of this disclosure, the term “toilet” may include a urine collection or receiving device such as a urinal, bidet, bowl, bucket, cup, etc., and thus, may all be used interchangeably.

A cartridge-based biofluid monitoring system and method of using same are disclosed. The biofluid monitoring system uses a biofluid cartridge configured to be removably inserted into a rotatable biofluid cartridge housing. The biofluid monitoring system may be used at a user's home or at other locations that allow for real-time biofluid monitoring to occur. The biofluid monitoring system allows for point-of-care detection and monitoring of a multitude of clinically relevant biological markers of current or changing health and disease states, by analyzing the user's biofluid.

Urinalysis monitoring is currently done in clinical settings to monitor health across a wide variety of conditions such as kidney disease, diabetes, and pregnancy. Urinary output is capable of both diagnosing previously unknown conditions and monitoring the progression of already known disorders. Unfortunately, due to the range of tests available and the previously costly and time-consuming methods of analysis, at-home urinary output monitoring is not currently commonplace.

This disclosure describes an at-home urinary analysis machine/system using cartridges with the ability to detect a multitude of clinically relevant markers. The biofluid output is analyzed for biological markers important in health and disease, in a real-life setting, with the potential to be used each time a person would like to analyze their biofluid. The biofluid monitoring system solves the problem of how to do portable/at-home, one-time or repeated monitoring of biofluid products of the human body, which would allow for the early detection and monitoring of diseases. All other systems that monitor biofluids (such as urine, for clinical urinalysis) currently require expensive, large lab equipment. The disclosed biofluid monitoring system will allow for non-invasive testing of biofluid samples.

Currently, colorimetric urine test strips are the only affordable on-market way to analyze biofluids such as urine. Colorimetric assay strips do not contain positive and negative controls, making precise detection of analyte levels impossible. Due to the nature of the strip, color bleed-through from neighboring sensors occurs frequently, confounding the assay's results. Additionally, modularity of the tests is not possible with one strip containing numerous, but in some cases medically unnecessary, testing measures.

In an embodiment, the biofluid monitoring system disclosed herein may be an all-in-one machine/system which allows for customization of urinary tests, test output detection and analysis, and sending the output to an application (which could be on a mobile phone or tablet) and/or a computer system/server for further analysis and/or storage of data.

The biofluid monitoring system is able to contain a user's biofluid (e.g., urine, blood, sweat, saliva) within a cartridge. The cartridge may be disposable after a single use, or may be reusable. Biofluid/Urine is placed into the storage chamber of each biofluid. A testing strip, storage chamber coating, or testing liquid (or other testing composition, for example, one that includes a color-changing dye) may be placed within the cartridge prior to the placement of the biofluid/urine. After the biofluid/urine makes contact with the testing strip/coating/liquid, the strip/coating/liquid would fluoresce a particular color. Then, a spectrometer is used to shine a light through the fluoresced liquid and the output light would be detected by a detector. The system therefore may allow for sequential and continuous operation, and is capable of obtaining a specific reading about the level of, for example, glucose, nitrites, salt, minerals, and/or vitamins in the urine.

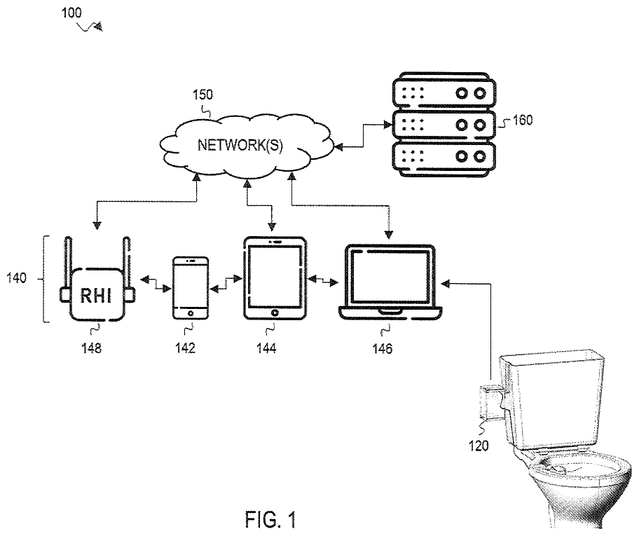

FIG. 1 is a diagram of a health ecosystem 100 according to an exemplary embodiment.

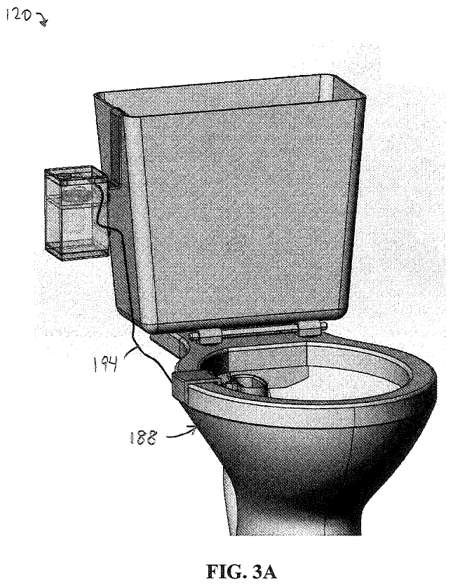

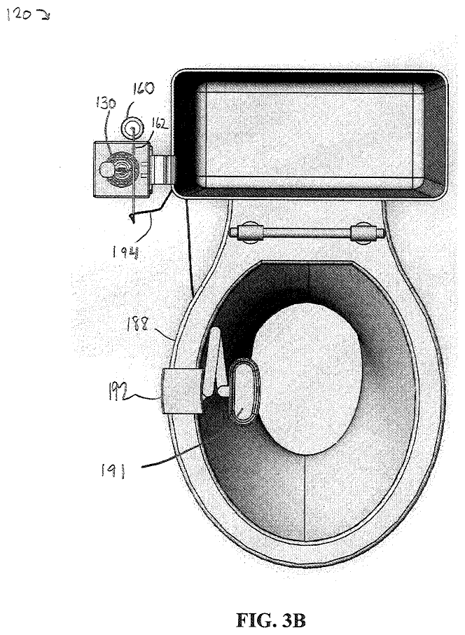

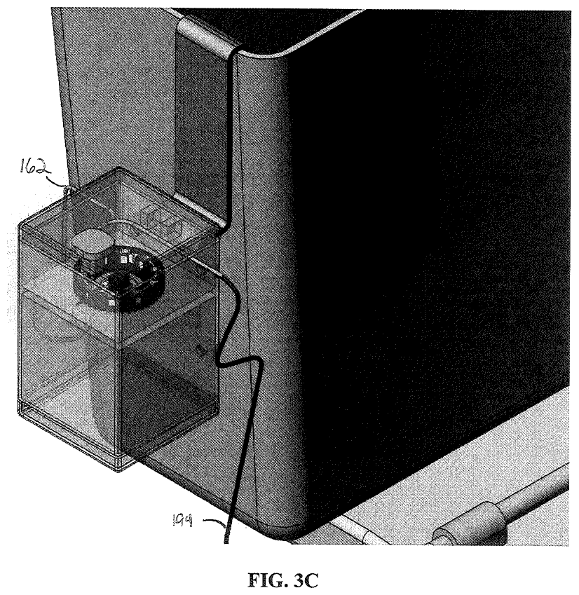

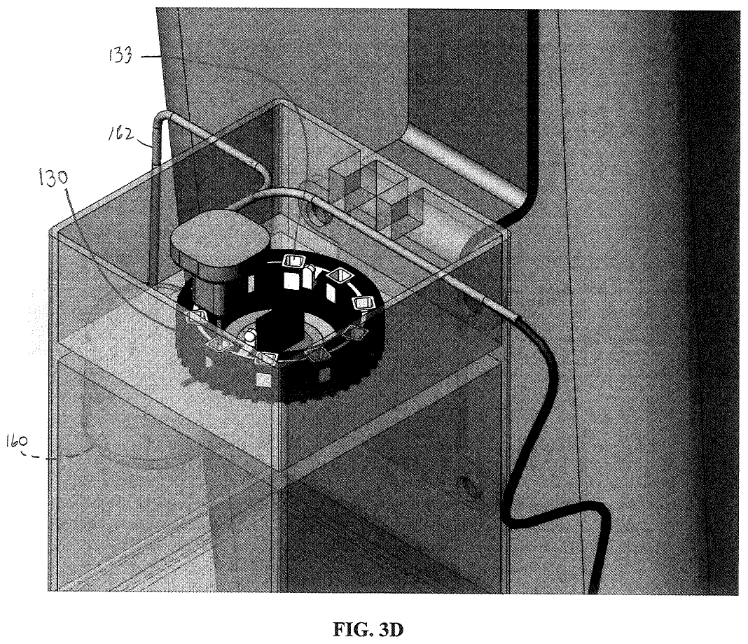

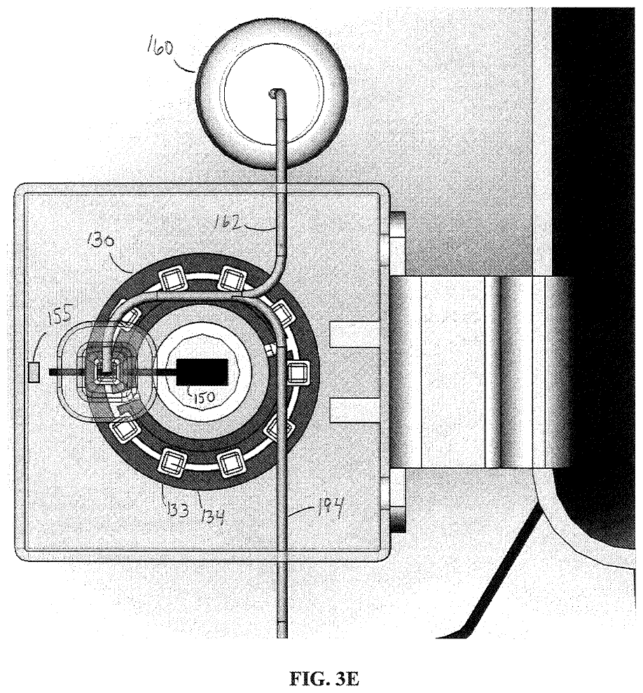

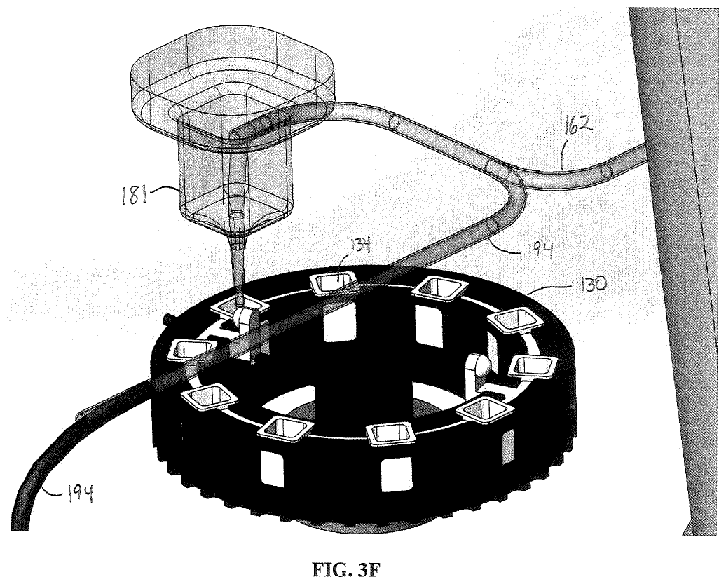

As shown in FIG. 1, the health ecosystem 100 includes a biofluid analyzer (such as biofluid monitoring system 120, for example as described in detail below with reference to FIGS. 3A-3F) that communicate with a server 160 via local computing devices 140 and one or more computer networks 150. The server 160 is a server that stores (or which as access to) health records and other health-related information.

The biofluid monitoring system 120, as described below, may be any device capable of analyzing biofluid to identify biological markers of current or changing health and disease states. For example, the biofluid monitoring system 120 may capture biofluid and dispense the captured biofluid (e.g., a predetermined amount of biofluid) into a chemically coated storage chamber of a disposable biofluid cartridge or in a disposable biofluid cartridge's (e.g., non-chemically coated) storage chamber housing a testing strip in contact with biofluid stored within the storage chamber. The biofluid may and the chemical coating (or testing strip) may initiate chemical reactions that cause color changes within the disposable biofluid cartridge that are indicative of biological markers. The biofluid monitoring system 120 may then measure those color changes (e.g., using a spectrometer) and output data indicative of those biological markers to a local computing device 140.

In preferred embodiments, the biofluid monitoring system 120 wirelessly communicates with the local computing devices 140 directly (e.g., using Bluetooth, Zigbee, etc.) or via a local area network (e.g., a Wi-Fi network). In other embodiments, the biofluid monitoring system 120 may transfer data using a wired connection (e.g., a USB cable) or by storing data in a removable storage device (e.g., a USB flash memory device, a microSD card, etc.) that can be removed and inserted into a local computing device 140.

The local computing devices 140 may include any hardware computing device having one or more hardware computer processors that perform the functions described herein. For example, the local computing devices 140 may include smartphones 142, tablet computers 144, personal computers 146 (desktop computers, notebook computers, etc.), etc. The local computing devices 140 may also include dedicated processing devices 148 (installed, for example, in hospitals or other clinical settings) that form local access points to wirelessly receive data from the biofluid monitoring system 120.

As described in detail below, the local computing devices 140 receive and process data from the biofluid monitoring system 120 and output the processed data to the server 160 via the one or more networks 150 (e.g., local area networks, cellular networks, the Internet, etc.). In some embodiments, the local computing devices 140 wirelessly communicate with each other, either via a local area network or using direct, wireless communication (e.g., via Bluetooth, Zigbee, etc.) to form a mesh network. Accordingly, in some embodiments, the biofluid monitoring system 120 may output data to a child data acquisition device (not shown), which forwards that data to a parent data acquisition device (not shown) that forwards the data to the server 160. The server 160 may be any hardware computing device having one or more hardware computer processors that perform the functions described herein.

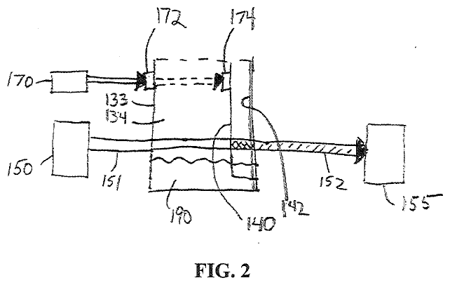

FIG. 2 is a drawing illustrating a schematic view of an exemplary overall workflow design of a biofluid monitoring system from a spectrometer light source 150, through a sample, to a detector 155.

FIG. 3A is a drawing illustrating an exemplary partially transparent biofluid monitoring system 120 for sterile collection and analysis of urine.

FIG. 3B is a drawing illustrating a plan view of the biofluid monitoring system 120 shown in FIG. 3A.

FIG. 3C is a drawing illustrating a enlarged perspective view of a portion of the biofluid monitoring system 120 shown in FIG. 3B. The figure shows, inter alia, a tube 194 for transport of biofluid into cartridges, as well as a cleaning fluid conduit 162 for the cleaning fluid for post-analysis cleansing.

FIG. 3D is a drawing illustrating a further enlarged view of a portion of the biofluid monitoring system shown in FIG. 3C. The removable biofluid cartridges 133 can be installed from above into the rotatable biofluid cartridge housing 130.

FIG. 3E is a drawing illustrating a plan view of the portion of the biofluid monitoring system shown in FIG. 3D. The spectrometer 150 is positioned in the center of the rotatable biofluid cartridge housing 130. The spectrometer 150 is in a fixed physical position, and the biofluid cartridges 133 are filled with the tube 194 and then rotated and each read by the spectrometer 150/detector 155. Following that, the biofluid cartridge 133 can be removed and then the system flushed with cleaning fluid from the cleaning fluid container 160 via the cleaning fluid conduit 162.

FIG. 3F is a drawing illustrating a further enlarged view of the rotatable biofluid cartridge housing 130 with the pipette 181 to fill the biofluid cartridges 133.

With reference to FIGS. 2-3F, a urine collection cup 191 (see FIG. 3B) is attached to toilet 188 via an extendable bracket mechanism 192. The urine collection cup 191 includes a drain (not shown) which is in communication with tube 194 which transports the biofluid/urine 190 from the urine collection cup 191 to the storage chambers 134 of the biofluid cartridges 133. That fast and efficient urine collection process allows a user to collect urine many times a day, if desired.

Testing strips in the form of, for example, graphene sheets, can optionally be used within a storage chamber 134. Graphene has the ability to have things added to it that will collect different proteins or samples within the biofluid. So, graphene is a type of multi-purpose tool or testing strip that can be used to indicate certain biological markers when configured to do so. A graphene sheet may be employed that has a moiety specific to pick-up a glucose nodule and will then change fluoresce when in contact with the biofluid dependent on how the graphene is created/employed.

A biofluid cartridge (i.e., involving a testing scenario via testing strip, storage chamber coating, or testing liquid) may be tailored for a user working out, for example, one who exercises often and who is concerned about their nutrition so the user can have nutrition gauged and the health of the user's muscles gauged. The system will inform the user of released chemicals from the muscles that signify that the user exerted themselves too hard, and may therefore have excessive muscle damage.

A biofluid cartridge (i.e., involving a testing scenario via testing strip, storage chamber coating, or testing liquid) may also or alternatively be employed that is tailored towards a user interested in family planning. In that scenario, the biofluid monitoring system would monitor a woman's menstrual cycle and/or whether the woman is pregnant.

A biofluid cartridge (i.e., involving a testing scenario via testing strip, storage chamber coating, or testing liquid) may also or alternatively be employed that is tailored towards a user that has diabetes. In that scenario, the biofluid monitoring system would monitor levels of, for example, glucose and/or ketone, and/or could monitor other criteria associated with liver and/or kidney functions.

A spectrometer 150 is positioned on a stationary platform that will allow one to target a variety of different biofluid cartridges 133 (with biofluid sample(s) contained therein) via rotation of the rotatable biofluid cartridge housing 130. A detector is further placed on the stationary platform or on another stationary platform (see FIG. 3E) with a portion of the rotatable biofluid cartridge housing 130 rotatably positioned between the spectrometer 150 and the detector 155. The detector 155 would send a digital representation of the light information (e.g., what wavelength is read) back to a storage device to store the light information. And then the light information is transmitted via Bluetooth or other wireless or wired connection to an application, server 160, or other remote computer system for further analysis.

With respect again to FIG. 2 and FIG. 3E, the rotatable biofluid cartridge housing 130 may be rotated to apply light 151 from the spectrometer 150 to individual biofluid cartridges 133, one at a time. Light 151 with different wavelengths may be applied to each biofluid cartridge 133. The light 151 from the spectrometer 150 will pass through a particular filter that narrows the range of light into the wavelength type which excites the liquid (indicative via the testing strip, storage chamber coating, or testing liquid) at a certain frequency so that it will only emit at a certain frequency for the detector 155 to detect. The targeting of one cartridge at a time makes the system more specific and receives the designated signal light with high efficiency and accuracy. By using the spectrometer's own focusing element, one can instruct which excitation wavelength to use. After the spectrometer diffracts the light through a prism, the spectrometer uses a slit member to pass light therethrough. The spectrometer itself is able to diffract the light and also then take the diffracted light and filter/use a specific wavelength.

In any of the embodiments described herein, certain components of the biofluid monitoring system 120 such as the barcode reader and/or QR code reader 170, spectrometer 150, detector 155, and rotatable biofluid cartridge housing 130, may be controlled via a CPU of one of the local computing devices 140 or of server 160 or of a separate server.

Embodiments of an exemplary method of using the cartridge-based biofluid monitoring system include:

1. Slot biofluid cartridge 133 into rotatable biofluid cartridge housing 130;

2. Urinate;

3. Transport urine from urine collection cup 191 to the storage chambers 134 of the biofluid cartridges 133;

4. Hit Go:
a. CPU (associated, for example, with one of the local computing devices 140 or of server 160 or of a separate server) receives barcode or QR code information (read by barcode reader or QR code reader 170) to determine an excitation test to be performed. The CPU then sends instructions to the spectrometer to perform a certain excitation test on the sample. Based on the chosen excitation test, if a particular testing strip, storage chamber coating, or testing liquid in a particular storage chamber 134 of a biofluid cartridge 133 is to be employed, the CPU would also send instructions to the motor (via a motor controller) to effect rotation of the rotatable biofluid cartridge housing 130 in order to directly align the particular testing strip, storage chamber coating, or testing liquid with the spectrometer 150 for the chosen excitation test of the biofluid within the particular storage chamber 134 to be performed;
b. Light emitted, sent through sample, excitation capture on detector, wavelength sent to an App running on a local computing device 140 for analysis;
c. Wavelength compared to reference sample;
d. Determination of concentration of analyte; and
e. Reporting of emission wavelength, concentration, and where analyte falls in health and disease states;

5. Rotatable biofluid cartridge housing 130 rotates to next position, and process outlined in step 4 begins anew for a different biofluid cartridge 133; and

6. Report data on App.

With further reference to FIGS. 2-3F, embodiments are directed to a cartridge-based biofluid monitoring system 120 including: a biofluid cartridge housing 130; and a biofluid cartridge 133 configured to be removably inserted into the biofluid cartridge housing 130. The biofluid cartridge 133 includes a storage chamber 134 which is configured to store biofluid 190.

In an embodiment, the biofluid cartridge housing 130 is a rotatable biofluid cartridge housing 130, and wherein the biofluid monitoring system 120 further comprises a plurality of biofluid cartridges 133 circularly distributed about the rotatable biofluid cartridge housing 130, and wherein the rotatable biofluid cartridge housing 130 is configured to rotate so that different types of analysis of the biofluid 190 in multiple storage chambers are sequentially performed. The rotatable biofluid cartridge housing 130 is connected to a motor that causes the rotatable biofluid cartridge housing 130 to rotate. The motor is controlled by a CPU which sends rotation instructions to the motor (via a motor controller) when analysis of the biofluid 190 in a subsequent storage chamber is to be performed in accordance with a reading of a barcode or QR code 172, 174 (described in more detail below). The CPU may, for example, be associated with one of the local computing devices 140 or of server 160 or of a separate server.

In an embodiment, the biofluid monitoring system 120 further comprises a tube 194 configured to transport the biofluid 190 to the storage chamber 134 of each of the biofluid cartridges 133 upon rotation of the rotatable biofluid cartridge housing 130.

In an embodiment, the biofluid monitoring system 120 further comprises a tube 194 and a toilet 188, wherein the tube 194 is configured to transport the biofluid 190 from the toilet 188 to the storage chamber 134 of each of the biofluid cartridges 133 upon rotation of the rotatable biofluid cartridge housing 130, and wherein the biofluid 190 is urine.

In an embodiment, the storage chamber 134 has a chemical coating 142, or the biofluid cartridge is further configured to hold a testing strip 140, in contact with biofluid 190 stored within the storage chamber 134, wherein the chemical coating 142 or the testing strip 140 is configured to initiate a chemical reaction that causes a color change within the storage chamber 134 that is indicative of a biological marker.

In an embodiment, the biofluid monitoring system 120 further comprises a spectrometer 150 (on a stationary platform) and a detector 155, wherein a portion of the rotatable biofluid cartridge housing 130 is positioned between the spectrometer 150 and the detector 155, and wherein the biofluid cartridge 133 is transmissible to light input 151 from the spectrometer and light output 152 to the detector 155 that has been transmitted through the chemically coated storage chamber 134 or the testing strip 140. The spectrometer 150 is positioned within a center of the rotatable biofluid cartridge housing 130.

In an embodiment, the biofluid monitoring system 120 further comprises a cleaning fluid container 160 and a cleaning fluid conduit 162 configured to dispense cleaning fluid from the cleaning fluid container 160 to the storage chamber 134 of each of the biofluid cartridges 133 subsequent analysis of the biofluid 190 and upon rotation of the rotatable biofluid cartridge housing 130.

In an embodiment, the biofluid monitoring system 120 further comprises cleaning fluid container 160 and a cleaning fluid conduit 162 configured to dispense cleaning fluid from the cleaning fluid container 160 to the rotatable biofluid cartridge housing 130 subsequent analysis of the biofluid 190.

In an embodiment, the biofluid monitoring system 120 further comprises cleaning fluid container 160 and a cleaning fluid conduit 162 connected to the tube 194, wherein the cleaning fluid conduit 162 is configured to dispense cleaning fluid from the cleaning fluid container 160 to the tube 194 subsequent analysis of the biofluid 190. In this embodiment, the biofluid cartridges 133 would be removable and disposable, so that the cleaning fluid would not need to clean the biofluid cartridges 133 themselves, but rather would need to clean just the tubing 194.

In an embodiment, the biofluid cartridge 133 comprises a barcode or QR code 172 and the biofluid monitoring system 120 further comprises a barcode reader or QR code reader 170 that reads the barcode or QR code 172, to identify a particular biofluid analysis to be performed by the biofluid monitoring system 120. The testing strip 140 can alternatively comprise a barcode or QR code 174. Using a barcode or QR code 172, 174 (or other type of identifying system) provides information as to which type (and/or number of) biofluid tests are to be performed by the biofluid monitoring system 120.

Embodiments are also directed to a method for using a cartridge-based biofluid monitoring system. The method includes providing a cartridge-based biofluid monitoring system including: a biofluid cartridge housing; and a biofluid cartridge comprising a storage chamber. The method also includes: removably inserting the biofluid cartridge into the biofluid cartridge housing; and storing biofluid in the storage chamber.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A cartridge-based biofluid monitoring system comprising:
a biofluid cartridge housing; and
a biofluid cartridge configured to be removably inserted into the biofluid cartridge housing, wherein the biofluid cartridge comprises a storage chamber;
wherein the storage chamber is configured to store biofluid.

2. The monitoring system of claim 1, wherein the biofluid cartridge housing is a rotatable biofluid cartridge housing, wherein the monitoring system further comprises a plurality of biofluid cartridges circularly distributed about the rotatable biofluid cartridge housing, and wherein the rotatable biofluid cartridge housing is configured to rotate so that different types of analysis of the biofluid in multiple storage chambers are sequentially performed.

3. The monitoring system of claim 2 further comprising a tube configured to transport the biofluid to the storage chamber of each of the biofluid cartridges upon rotation of the rotatable biofluid cartridge housing.

4. The monitoring system of claim 2 further comprising a tube and a toilet, wherein the tube is configured to transport the biofluid from the toilet to the storage chamber of each of the biofluid cartridges upon rotation of the rotatable biofluid cartridge housing, and wherein the biofluid is urine.

5. The monitoring system of claim 2, wherein the storage chamber has a chemical coating, or the biofluid cartridge is further configured to hold a testing strip, in contact with biofluid stored within the storage chamber, and wherein the chemical coating or the testing strip is configured to initiate a chemical reaction that causes a color change within the storage chamber that is indicative of a biological marker.

6. The monitoring system of claim 5 further comprising a spectrometer and a detector, wherein a portion of the rotatable biofluid cartridge housing is positioned between the spectrometer and the detector, and wherein the biofluid cartridge is transmissible to light input from the spectrometer and light output to the detector that has been transmitted through the chemically coated storage chamber or the testing strip.

7. The monitoring system of claim 6, wherein the spectrometer is positioned within a center of the rotatable biofluid cartridge housing.

8. The monitoring system of claim 2 further comprising a cleaning fluid container and a cleaning fluid conduit configured to dispense cleaning fluid from the cleaning fluid container to the storage chamber of each of the biofluid cartridges subsequent analysis of the biofluid and upon rotation of the rotatable biofluid cartridge housing.

9. The monitoring system of claim 3 further comprising cleaning fluid container and a cleaning fluid conduit connected to the tube, wherein the cleaning fluid conduit is configured to dispense cleaning fluid from the cleaning fluid container to the tube subsequent analysis of the biofluid.

10. The monitoring system of claim 2, wherein the biofluid cartridge comprises a barcode or QR code and the monitoring system further comprises a barcode reader or QR code reader, to identify a particular biofluid analysis to be performed by the monitoring system.

11. A method for using a cartridge-based biofluid monitoring system, the method comprising:
providing a cartridge-based biofluid monitoring system comprising:
a biofluid cartridge housing; and
a biofluid cartridge comprising a storage chamber;
removably inserting the biofluid cartridge into the biofluid cartridge housing; and
storing biofluid in the storage chamber.

12. The method of claim 11, wherein the biofluid cartridge housing is a rotatable biofluid cartridge housing, wherein the monitoring system further comprises a plurality of biofluid cartridges circularly distributed about the rotatable biofluid cartridge housing, and wherein the method further comprises rotating the rotatable biofluid cartridge housing so that different types of analysis of the biofluid in multiple storage chambers are sequentially performed.

13. The method of claim 12, wherein the monitoring system further comprises a tube, and wherein the method further comprises transporting, via a tube, the biofluid to the storage chamber of each of the biofluid cartridges upon rotation of the rotatable biofluid cartridge housing.

14. The method of claim 12, wherein the monitoring system further comprises a tube and a toilet, wherein the method further comprises transporting, via the tube, the biofluid from the toilet to the storage chamber of each of the biofluid cartridges upon rotation of the rotatable biofluid cartridge housing, and wherein the biofluid is urine.

15. The method of claim 12, wherein the storage chamber has a chemical coating, or the biofluid cartridge holds a testing strip, in contact with biofluid stored within the storage chamber, and wherein the chemical coating or the testing strip initiates a chemical reaction that causes a color change within the storage chamber that is indicative of a biological marker.

16. The method of claim 15, wherein the monitoring system further comprises a spectrometer and a detector, wherein a portion of the rotatable biofluid cartridge housing is positioned between the spectrometer and the detector, and wherein the biofluid cartridge is transmissible to light input from the spectrometer and light output to the detector that has been transmitted through the chemically coated storage chamber or the testing strip.

17. The method of claim 16, wherein the spectrometer is positioned within a center of the rotatable biofluid cartridge housing.

18. The method of claim 12, wherein the monitoring system further comprises a cleaning fluid container and a cleaning fluid conduit that dispenses cleaning fluid from the cleaning fluid container to the storage chamber of each of the biofluid cartridges subsequent analysis of the biofluid and upon rotation of the rotatable biofluid cartridge housing.

19. The method of claim 13, wherein the monitoring system further comprises a cleaning fluid container and a cleaning fluid conduit connected to the tube, wherein the cleaning fluid conduit dispenses cleaning fluid from the cleaning fluid container to the tube subsequent analysis of the biofluid.

20. The method of claim 12, wherein the biofluid cartridge comprises a barcode or QR code and the monitoring system further comprises a barcode reader or QR code reader, to identify a particular biofluid analysis to be performed by the monitoring system.
